(19) United States
(12) Patent Application Publication
(10) Pub. No.: US 2011/0083670 A1
(43) Pub. Date: Apr. 14, 2011

(54) BREATHING APPARATUS AND ASSOCIATED METHODS OF USE

(76) Inventor: Alexander J. Walacavage, Lansing, MI (US)

(21) Appl. No.: 12/902,242

(22) Filed: Oct. 12, 2010

Related U.S. Application Data
(60) Provisional application No. 61/250,807, filed on Oct. 12, 2009.

(51) Int. Cl. A61M 16/20; A61M 16/06

(52) U.S. Cl. 128/205.12; 128/205.24

(57) ABSTRACT

A breathing apparatus including a mask body adapted to sealingly cover at least a portion of a face of an individual, a securement member for operatively associating the mask body with the face of the individual, at least one inlet port providing communication of fluids into mask body, and at least one valve providing a uni-directional path for the communication of fluids through the mask body.

### CROSS-REFERENCE TO RELATED APPLICATION(S)

This application claims the benefit of U.S. Provisional Application Ser. No. 61/250,807, filed Oct. 12, 2009, entitled “Breathing Apparatus,” which is hereby incorporated herein by reference in its entirety, including all references cited therein.

### BACKGROUND OF THE INVENTION

1. Field of the Invention

The present invention relates in general to a breathing apparatus and, more particularly, to a breathing apparatus which is adapted to sealingly cover the mouth and nose of an individual and to filter the incoming air and/or exhaled breath of the individual.

2. Background Art

Breathing apparatuses have been known in the art for years, and are the subject of numerous patents, including: U.S. Pat. No. 5,884,624 entitled “Respiratory mask facial seal,” U.S. Pat. No. 4,770,169 entitled “Anesthetic mask,” U.S. Pat. No. 3,330,273 entitled “Oro-nasal face mask with improved sealing cuff,” U.S. Pat. No. 2,931,356 entitled “Oxygen mask having detachable face seal cushion,” United States Patent Application Publication Number 2008/0006276 entitled “Face mask, in particular inhalation mask for use with a therapeutic nebulizer,” and United States Patent Application Publication Number 2008/0230068 entitled “Ventilation mask with continuous seal connected by resilient cushion,”—all of which are hereby incorporated herein by reference in their entirety including the references cited therein.

U.S. Pat. No. 5,884,624 appears to disclose a respiratory mask and, more particularly, a respiratory mask having flexible seals adapted to receive portions of a user's face for preventing leakage of gas being supplied to the user.

U.S. Pat. No. 4,770,169 appears to disclose an improved anesthetic scavenging facemask having a scavenging channel running along the perimeter of the mask. The mask is provided with an anesthetic gas inlet and a vacuum outlet. The scavenging channel is connected to the vacuum outlet.

U.S. Pat. No. 3,330,273 appears to disclose an oro-nasal facemask for respiratory use, and more particularly to a mask embodying an improved sealing cuff for effecting a seal between the perimeter of the mask and the face and for cushioning the force exerted by the mask against the face.

U.S. Pat. No. 2,931,356 appears to disclose a gas distribution mask assembly and more particularly to an improved face seal unit mounted on the peripheral edge of the mask substantially conforming to the configuration of that portion of the wearers face over which the mask is adapted to be worn.

United States Patent Application Publication Number 2008/0006276 appears to disclose a face mask, in particular an inhalation mask for use with a therapeutic nebulizer, having a main mask body that includes a first circumferential region, which is designed for the application of the mask to a user's face, and a second region for connection to, in particular, a therapeutic nebulizer, wherein a section of the first region is designed for application to an upper nasal region of the face and comprises two sealing lips connected in a series. The two sealing lips, which are connected in series are formed by a first inward-facing sealing lip and a second outward-facing sealing lip.

United States Patent Application Publication Number 2008/0230068 appears to disclose a respiratory mask having a sealing flange connected to a mask body by a sinusoidal shaped cushioning member. The cushioning member resiliently compresses when the sealing flange is pressed against the face of a wearer to ensure an airtight seal between the sealing flange and the face of the wearer. The cushioning member functions as a spring, compressing to allow the sealing member to better conform to the face of the wearer but resiliently urging the sealing flange outward against the face of the wearer to prevent leaks.

While the above-identified patents and publications do appear to provide breathing apparatuses, their configurations remain non-desirous and/or problematic inasmuch as, among other things, none of the above-identified apparatuses appear to be configured to effectively provide a path for the uni-directional communication of fluids inhaled/exhaled by the user—among other things.

It is therefore an object of the present invention to provide a breathing apparatus, which, among other things, remedies the aforementioned detriments and/or complications associated with the use of the above-identified, conventional breathing apparatuses. It is further therefore an object of the present invention to provide a breathing apparatus which is adapted to sealingly cover the mouth and nose of an individual and to filter the incoming air and/or exhaled breath of the individual. It is yet a further object of the present invention to provide a breathing apparatus which regulates temperature and/or acts as a cooling mask, as well as facilitates self-fit and function testing.

These and other objects of the present invention will become apparent in light of the present specification, claims, and drawings.

### SUMMARY OF THE INVENTION

In one embodiment, the present invention is directed to a breathing apparatus that includes: (a) a mask body adapted to sealingly cover at least a portion of a face of an individual; (b) a securement member for operatively associating the mask body with the face of the individual; (c) at least one inlet port providing communication of fluids into the mask body; and (d) at least one valve providing a uni-directional path for the communication of fluids through the mask body.

In an additional embodiment, the mask body includes a seal disposed along a peripheral edge thereof.

In another embodiment, the securement member includes an elastomeric strap forming at least one loop configured to overlap at least a portion of the mask body and at least a portion of the head of the individual.

In yet another embodiment, the mask body is adapted to cover at least one of the nose and mouth of the individual.

In an alternate embodiment, the seal includes an air filled cushion.

In an additional embodiment, the air filled cushion includes a valve for varying the pressure within the air filled cushion.

In another embodiment, the at least one valve includes a diaphragm check valve.

In yet another embodiment, the breathing apparatus further includes a filter associated with the at least one valve for purifying fluids communicating through the at least one valve.

In an additional embodiment, the breathing apparatus further includes a filter associated with the at least one inlet port for purifying the fluids entering the mask body.

In an alternate embodiment, the filter includes a disposable filter assembly having a tubular body for retaining a filter, the tubular body adapted to releaseably connect to the tubular stem extending from the mask body.

In yet another embodiment, the at least one valve is associated with a tubular stem extending from the mask body.

In one embodiment, the present invention is directed to a breathing apparatus that includes: (a) a mask body adapted to sealingly cover at least a portion of a face of an individual; (b) a securement member for operatively associating the mask body with the face of the individual; (c) at least one inlet valve providing a uni-directional path for the communication of fluids into the mask body; and (d) at least one outlet valve providing a uni-directional path for the communication of fluids out of the mask body.

In an additional embodiment, the at least one inlet valve and the at least one outlet valve each include a diaphragm check valve.

### BRIEF DESCRIPTION OF THE DRAWINGS

Certain embodiments of the present invention are illustrated by the accompanying figures. It will be understood that the figures are not necessarily to scale and that details not necessary for an understanding of the invention or that render other details difficult to perceive may be omitted. It will be further understood that the invention is not necessarily limited to the particular embodiments illustrated herein.

The invention will now be described with reference to the drawings wherein:

FIG. 1 of the drawings is a perspective view of a portion of a breathing apparatus, constructed in accordance with the present invention;

FIG. 2 of the drawings is a perspective view of a portion of a breathing apparatus showing, among other things, a filter assembly connected to a tubular divider, which is associated with an inlet port of a mask body;

FIG. 3 of the drawings is a perspective view of a portion of a breathing apparatus showing, among other things, a filter assembly connected to an outlet port of a mask body; and

FIG. 4 of the drawings is a perspective view of a breathing apparatus associated with the face of an individual and secured around the head of the individual.

### DETAILED DESCRIPTION OF THE INVENTION

While this invention is susceptible of embodiment in many different forms, there is shown in the drawings and described herein in detail several specific embodiments with the understanding that the present disclosure is to be considered as an exemplification of the principles of the invention and is not intended to limit the invention to the embodiments illustrated.

It will be understood that like or analogous elements and/or components, referred to herein, may be identified throughout the drawings with like reference characters.

In accordance with the present invention, the breathing apparatus disclosed herein provides protection against foreign matter such as bacterial and/or viral pathogens and/or other air-born particulates. The breathing apparatus reduces and/or eliminates the need for annual testing for proper fitting as is common with typical breathing apparatuses. The breathing apparatus may be used in inhalation mode or exhalation mode depending on the infection status of the individual. Additionally, the breathing apparatus substantially regulates temperature within the breathing apparatus relative to commonly utilized apparatuses in that commonly utilized apparatuses trap the exhaled breath of the individual, which may lead to condensation and/or heat build-up within the mask.

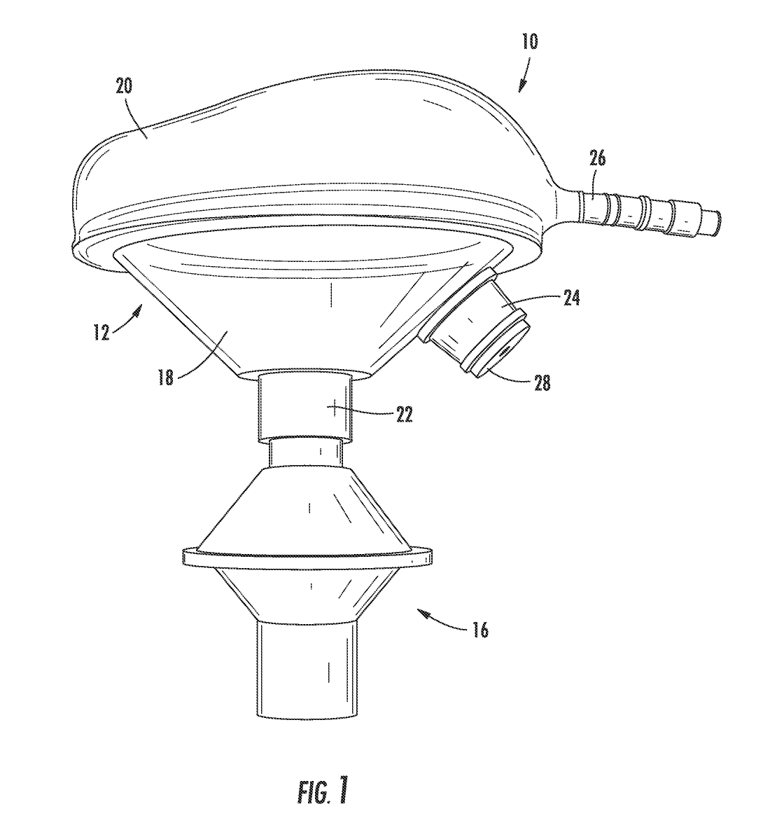

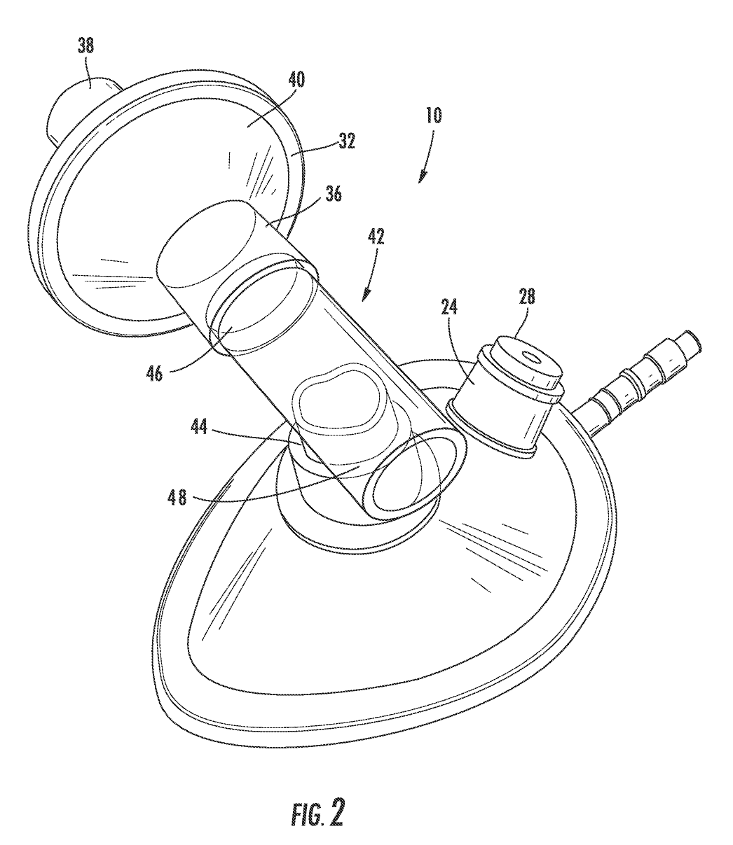

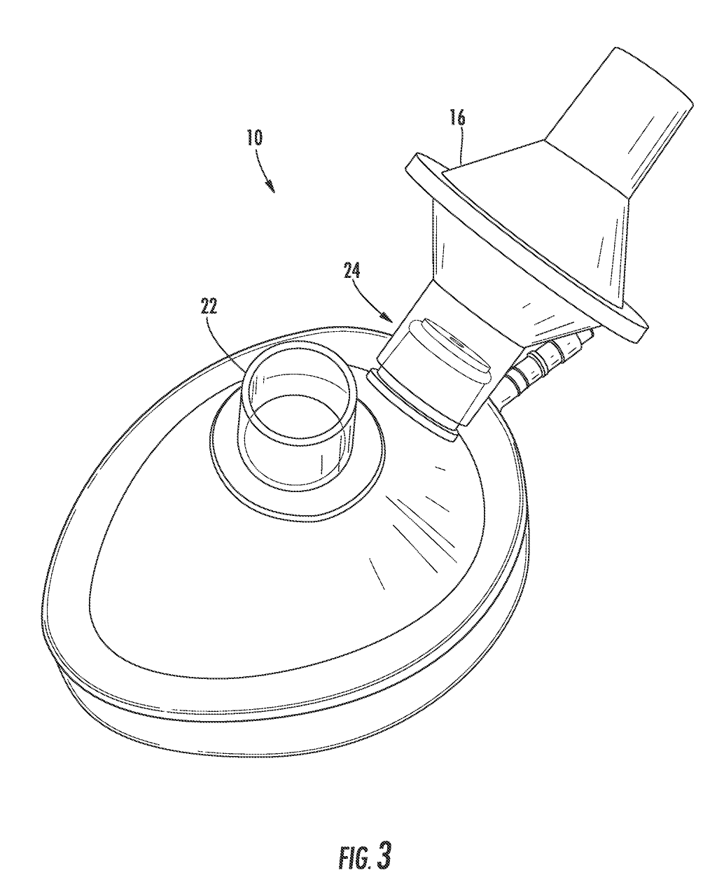

Referring now to the collective drawings (i.e., FIGS. 1-4), breathing apparatus 10 generally comprises mask 12, strap 14, and one or more filter assemblies 16. Mask 12 generally comprises mask body 18, seal 20, inlet port 22, and outlet port 24. Mask body 18 is shaped to sealingly cover at least a portion of an individual's face, such as the individual's mouth or nose. More specifically, mask body 18 is sized and configured such that when mask body 18 is positioned against the face of the wearer, a peripheral edge of seal 20 generally extends across the bridge of the nose, down the sides of the nose, downward across the cheeks of the wearer on opposite sides of the mouth, and abuts and/or sealingly overlaps the chin of the individual. Mask body 18 is preferably fabricated from a resilient material such as a plastic or resin, although other suitable materials that would be known to one of ordinary skill in the art having the present disclosure before them are likewise contemplated for use in accordance with the present invention. It will be understood that mask body 18 is preferably fabricated from a material that is generally flexible and/or non-flexible—depending upon the anticipated conditions of use.

Seal 20 is disposed continuously along the peripheral edge of mask body 18 to prevent foreign matter such as viral and/or bacterial pathogens and/or other particulate matter such as dust from the atmosphere from entering mask body 18.

Seal 20 may include any one of a number of sealing devices commonly utilized for facemasks such as air filled cushions or rubber gaskets, although any number of sealing devices which would be known to one of ordinary skill in the art having the present disclosure before them are likewise contemplated for use in accordance with the present invention. In one embodiment, seal 20 includes an air filled cushion having at least one port 26 for controlling the level of air within the air filled cushion. As facial characteristics such as size and proportion vary from individual to individual, the adjustability of the air filled cushion allows mask 12 to be adapted to fit a variety of individuals for a plurality of conditions.

Additionally, seal 20 may cooperate with at least one of inlet port 22 and outlet port 24 to facilitate the induction of vacuum pressure within mask body 18 such that mask body 18 can be sealingly interfaced with the face of the individual and further prevent the passage of foreign matter. A vacuum can be induced by first pressing mask body 18 to the face of the individual such that seal 20 is disposed around the nose and mouth of the individual. Next, at least one of inlet port 22 and outlet port 24 are sealed off, and the individual inhales through the nose and/or mouth to create negative pressure within mask body 18 causing seal 20 to conform to the face of the individual, securing mask body 18 to the face of the individual.

In one embodiment, inlet port 22 is a tubular member providing a path for fluid communication from outside mask body 18, inwardly. Inlet port 22 is formed into mask body 18 and extends from mask body 18 opposite seal 20. Inlet port 22 is sized to cooperate with various medical implements (not shown) such as a resuscitation bag or tubing providing oxygen or other medical gases. Additionally, both inlet port 22 and outlet port 24 are configured to receive and retain at least a portion of filter assembly 16 as will be discuss in greater detail infra.

Similarly to inlet port 22, outlet port 24 preferably comprises a tubular member providing a path for fluid communication, but in contrast to inlet port 22, outlet port 24 provides a uni-directional path for fluid communication from inside mask body 18 outwardly from mask body 18. That is, outlet port 24 functions as a one-way valve for communicating exhaled breath of the individual outwardly from mask body 18. In one embodiment, outlet port 24 includes flap 28 covering the distal end of outlet port 24. Flap 28 is secured to strut (not shown) that partially covers the distal end of outlet port 24. In a sealed position (e.g., when the individual is inhaling), flap 28 completely covers the distal end of outlet port 24 to prevent communication of fluids therethrough. According to exemplary embodiments, flap 28 may include a diaphragm valve that is fabricated from a thin and elastic material such as a rubber that allows flap 28 to deflect to permit exhaled breath of the individual to exit mask body 18. While outlet port 24 has been disclosed as including flap 28, it will be understood that many different uni-directional or “check” valves that provide one-way communication of materials which would be known to one of ordinary skill in the art having the present disclosure before them are likewise contemplate for use in accordance with the present invention.

Filter assembly 16 generally includes housing 32 and filter (not shown). Housing 32 is fabricated having inlet port 36 and outlet port 38 which are spaced apart from one another by filter retaining portion 40 sized to receive and retain the filter. Fluid communicating from inlet port 36 to outlet port 38 or vice-versa passes through the filter that operates to substantially capture and/or neutralize foreign matter such as bacterial and/or viral pathogens. Additionally, filter assembly 16 may be configured to capture and/or reduce CO₂ emissions from the exhaled breath of the individual when filter assembly 16 is joined to outlet port 24 of mask body 18. Inlet port 36 and outlet port 38 are configured to cooperate with inlet port 22 of mask body 18 or various medical devices (not shown). Furthermore, filter assembly 16 may be positioned between inlet port 22 of mask body 18 and various medical implements.

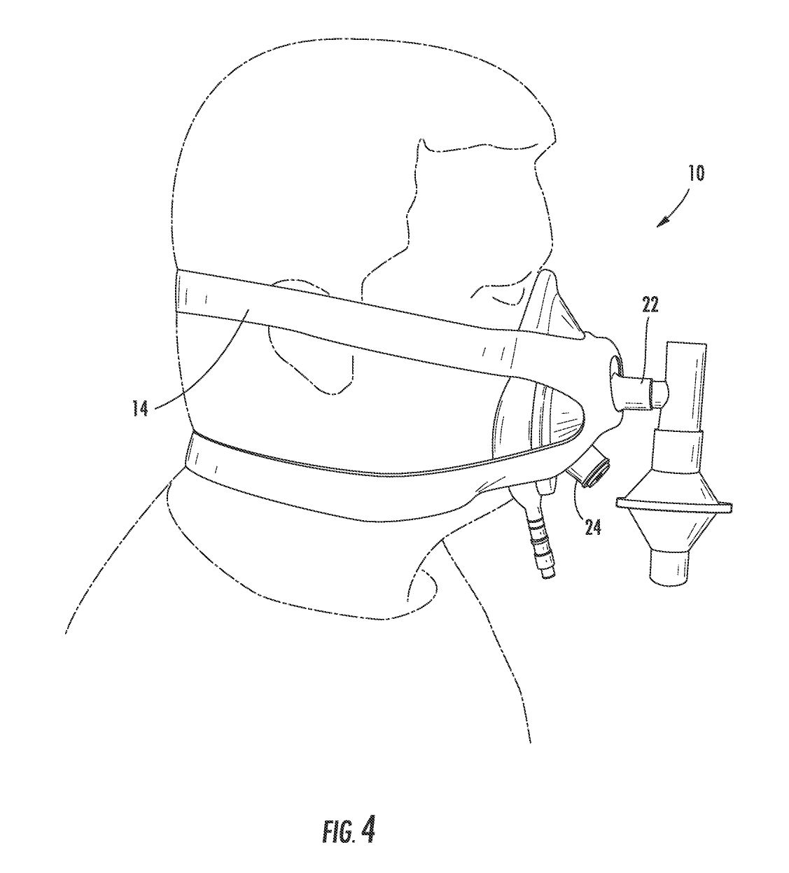

Additionally, as depicted in FIG. 4, filter assembly 16 may attach to outlet port 24 of mask body 18 to capture and/or neutralize bacterial and/or viral pathogens contained in exhaled breath.

Breathing apparatus 10 may also optionally include a T-shaped tubular divider 42 having first end 44, second end 46, and third end 48. Divider 42 is configured to cooperate with at least one of inlet port 22 and outlet port 24 of mask body 18. In one embodiment, first end 44 of divider 42 is inserted into inlet port 22 of mask body 18 and filter assembly 16 is connected to second end 46. Third end 48 of divider 42 may optionally cooperate with a medical implement as discussed above or another filter assembly 16. Divider 42 may likewise be connected to outlet port 24 of mask body 18 such that two filter assemblies 16 may be associated with outlet port 24 of mask body 18.

Strap 14 includes at least one elastic strap sized to cooperate with at least a portion of breathing apparatus 10 and fit around the head and/or neck of the individual to retain breathing apparatus 10 on the face of the individual. In one embodiment, strap 14 includes two loops for overlapping different sections of the individual's head. In another embodiment, strap 14 includes an aperture (not shown) for receiving inlet port 22 of mask body 18. Furthermore, strap 14 is sized to compress breathing apparatus 10 against the face of the individual to further sealingly cover the individual's face and prevent breathing apparatus 10 from unwanted removal.

Potential alternative uses for breathing apparatus 10 include athletic applications such as for runners performing in cold/hot environments. A tube (not shown) may be attached to inlet port 22 that is inserted under the runners clothing that pre-heats or pre-cools the air prior to introduction through inlet port 22.

Also, it will be understood that because filter assemblies 16 are replaceable and mask body 18 can be sterilized, breathing apparatus 10 can be sanitized and reused as needed.

In operation, to prepare for use, the individual inflates seal 20 (e.g., air cushion) of mask body 18 via port 26 to a desired comfort level. It will be understood that port 26 is optional and that seal 20 may comprise a pre-inflated cushion void of the same. Next, strap 14 is configured for use by inserting inlet port 22 through aperture (not shown) of mask body 18. Next, the individual positions mask body 18 over the nose and mouth of their face and positions strap 14 around their head, adjusting for fit and comfort as needed. Mask body 18 is optionally further secured to the individual's face by covering inlet port 22 of mask body 18 and inhaling to create a vacuum between seal 20 and the face of the individual. Next, one or more filter assemblies 16 are associated with at least one of inlet port 22 and outlet port 24 of mask body 18. Filter assemblies 16 can be removed and replaced as needed.

The foregoing description merely explains and illustrates the invention and the invention is not limited thereto except insofar as the appended claims are so limited, as those skilled in the art who have the disclosure before them will be able to make modifications without departing from the scope of the invention.

What is claimed and desired to be secured by Letters Patent of the United States is:

1. A breathing apparatus, comprising:
a mask body adapted to sealingly cover at least a portion of a face of an individual;
a securement member for operatively associating the mask body with the face of the individual;
at least one inlet port providing communication of fluids into mask body; and
at least one valve providing a uni-directional path for the communication of fluids through the mask body.

2. The breathing apparatus according to claim 1, wherein the mask body includes a seal disposed along a peripheral edge thereof.

3. The breathing apparatus according to claim 1, wherein the securement member includes an elastomeric strap forming at least one loop configured to overlap at least a portion of the mask body and at least a portion of the head of the individual.

4. The breathing apparatus according to claim 1, wherein the mask body is adapted to cover at least one of the nose and mouth of the individual.

5. The breathing apparatus according to claim 1, wherein the seal includes an air filled cushion.

6. The breathing apparatus according to claim 5, wherein the air filled cushion includes a valve for varying the pressure within the air filled cushion.

7. The breathing apparatus according to claim 1, wherein the at least one valve includes a diaphragm check valve.

8. The breathing apparatus according to claim 1, further comprising a filter associated with the at least one valve for purifying fluids communicating through the at least one valve.

9. The breathing apparatus according to claim 1, further comprising a filter associated with the at least one inlet port for purifying the fluids entering the mask body.

10. The breathing apparatus according to claim 9, wherein the filter includes a disposable filter assembly having a tubular body for retaining a filter, the tubular body adapted to releaseably connect to the tubular stem extending from the mask body.

11. The breathing apparatus according to claim 1, wherein the at least one valve is associated with a tubular stem extending from the mask body.

12. A breathing apparatus, comprising:
a mask body adapted to sealingly cover at least a portion of a face of an individual;
a securement member for operatively associating the mask body with the face of the individual;
at least one inlet valve providing a uni-directional path for the communication of fluids into the mask body; and
at least one outlet valve providing a uni-directional path for the communication of fluids out of the mask body.

13. The breathing apparatus according to claim 12, wherein the mask body includes a seal disposed along a peripheral edge thereof.

14. The breathing apparatus according to claim 12, wherein the securement member includes an elastomeric strap forming at least one loop configured to overlap at least a portion of the mask body and at least a portion of the head of the individual.

15. The breathing apparatus according to claim 12, wherein the mask body is adapted to cover at least one of the nose and mouth of the individual.

16. The breathing apparatus according to claim 12, wherein the seal includes an air filled cushion.

17. The breathing apparatus according to claim 16, wherein the air filled cushion includes a valve for varying the pressure within the air filled cushion.

18. The breathing apparatus according to claim 12, wherein the at least one inlet valve and the at least one outlet valve each include a diaphragm check valve.

19. The breathing apparatus according to claim 12, further comprising a filter associated with the at least one outlet valve for purifying fluids communicating through the at least one valve.

20. The breathing apparatus according to claim 12, further comprising a filter associated with the at least one inlet valve for purifying the fluids entering the mask body.
